(19) United States
(12) Patent Application Publication
(10) Pub. No.: US 2016/0053028 A1
(43) Pub. Date: Feb. 25, 2016

(54) MEDIUM ADDITIVE FOR CELL CULTURE

(71) Applicant: DAISO CO., LTD., Osaka (JP)

(72) Inventors: Masahide KIDO, Osaka-shi, Osaka (JP); Keiko MATONO, Osaka-shi, Osaka (JP); Hideaki IDOGAKI, Osaka-shi, Osaka (JP)

(73) Assignee: DAISO CO., LTD., Osaka-shi, Osaka (JP)

(21) Appl. No.: 14/780,315

(22) PCT Filed: Mar. 27, 2014
(86) PCT No.: PCT/JP2014/058784

§ 371 Date: Sep. 25, 2015

(30) Foreign Application Priority Data
Mar. 29, 2013 (JP) 2013-073019
Jul. 9, 2013 (JP) 2013-143357

(51) Int. Cl. C08B 37/00; C07K 16/00

(52) U.S. Cl. CPC C08B 37/0024; C07K 16/00

(57) ABSTRACT

Provided are a composition for enhanced production of a useful substance in cells containing a polysaccharide, a medium for cell culture containing a polysaccharide, and a process for preparing a useful substance including culturing cells in a medium for cell culture containing a polysaccharide.

### TECHNICAL FIELD

This application claims priority to and the benefit of Japanese Patent Application No. 2013-73019 filed Mar. 29, 2013 and Japanese Patent Application No. 2013-143357 filed Jul. 9, 2013, and the entire contents of which are incorporated herein by reference.

The present invention relates to a medium additive for cell culture, a medium for cell culture comprising said additive, and use of said additive and said medium for the manufacture of a useful substance.

### BACKGROUND ART

Proteins and peptides are artificially produced by a recombinant technique. Especially, a protein useful as a medicine such as humanized antibody and human antibody is produced in cultured cells in the pharmaceutical field.

A serum medium with a bovine serum has mainly been used as a conventional medium for cell culture. Such serum medium has a risk such as being contaminated by a pathogen during the production of a medicine, and thus a serum-free medium containing no serum has been required. However, a serum contains various growth factors involved in cell growth, and thus a simple elimination of a serum results in the significant suppression of the cell growth or protein productivity. Thus, a serum replacement such as insulin, transferrin, and sodium selenite is used in many cases. However, a serum-free medium, especially a medium consisting of substances having a defined chemical composition, i.e., a Chemically defined medium, shows lower cell growth or protein productivity as compared to a conventional serum medium. A protein hydrolysate such as yeast or soybean hydrolysate is used to improve the cell growth or protein productivity, and known to highly effectively promote the cell growth (Patent Literature 1). However, a protein hydrolysate is a composite substance which may contain many undefined ingredients, and thus is not preferable as the raw materials of pharmaceutical preparation in view of safety.

### CITATION LIST

### Patent Literature

Patent Literature 1: JP2002-520014A

### SUMMARY OF THE INVENTION

The present invention provides a medium additive for cell culture, a medium for cell culture comprising said additive, and use of said additive and said medium for the manufacture of a useful substance.

The present inventors have found that a polysaccharide increases the production of a useful substance such as protein in cells, and completed the present invention.

An aspect of the present invention provides a composition for enhanced production of a useful substance in cells comprising a polysaccharide.

An aspect of the present invention provides a medium for cell culture comprising a polysaccharide.

An aspect of the present invention provides a process for preparing a useful substance comprising culturing cells in a medium for cell culture comprising a polysaccharide.

### DETAILED DESCRIPTION OF THE INVENTION

The present invention provides, for example, a composition for enhanced production of a useful substance in cells comprising a polysaccharide; a medium for cell culture comprising a polysaccharide; and a process for preparing a useful substance comprising culturing cells in a medium for cell culture comprising a polysaccharide.

The polysaccharide which may be used in the present invention may be a straight chain polysaccharide containing a main chain only or a polysaccharide containing a main chain and a side chain. The polysaccharide which may be used in the present invention may be one type of straight chain polysaccharide, a mixture of two or more types of straight chain polysaccharides, one type of polysaccharide containing a main chain and a side chain, a mixture of two or more types of polysaccharides containing a main chain and a side chain, or a mixture thereof. The straight chain of a straight chain polysaccharide contains linked hexose residues, and the main chain of a polysaccharide containing a main chain and a side chain contains linked hexose residues. The linked hexose residues may be linked through any binding mode between carbon at 1-position and carbon at 3-position, carbon at 1-position and carbon at 4-position, or carbon at 1-position and carbon at 6-position. The binding mode between carbon at 1-position and carbon at 3-position includes β1,3 linkage and α1,3 linkage, the binding mode between carbon at 1-position and carbon at 4-position includes β1,4 linkage and α1,4 linkage, and the binding mode between carbon at 1-position and carbon at 6-position includes β1,6 linkage and α1,6 linkage. These α1,3 linkage, β1,3 linkage, α1,4 linkage, β1,4 linkage, α1,6 linkage, and β1,6 linkage are glycoside linkages, which are formed between carbons at 1-position and 3-position, carbons at 1-position and 4-position, or carbons at 1-position and 6-position via an oxygen atom.

The main chain containing linked hexose residues may further contain one or more side chains containing a hexose residue linked at the 2-position, 3-position, or 6-position. In such a case, the side chain may be constructed from two or more hexose residues linked with each other or one hexose residue, and one or more types of hexose may construct the side chain. The binding mode between a main chain and a side chain may be at least one binding mode selected from the group consisting of α1,2 linkage, α1,3 linkage, α1,6 linkage, β1,2 linkage, β1,3 linkage, and β1,6 linkage.

The hexose which constructs the polysaccharide which may be used in the present invention is a sugar containing six carbon atoms. Examples of the hexose include glucose, mannose, galactose, fucose, allose, altrose, gulose, idose, talose, rhamnose, fuculose, glucosamine, galactosamine, glucuronic acid, galacturonic acid, mannuronic acid, and a derivative thereof such as salt, ester, ether, and amide. The hexose may be a sugar containing an aldehyde group or an aldohexose. The hexose may be a deoxy sugar, a sugar containing carboxylic acid, or an amino sugar. Examples of the deoxy sugar include D-deoxyribose, L-fucose, L-rhamnose, D-allomethylose, D-quinovose, D-antiarose, D-talomethylose, L-talomethylose, D-digitalose, D-digitoxose, D-cymarose, tyvelose, abequose, paratose, colitose, and ascarylose. Examples of the sugar containing carboxylic acid include glucuronic acid, galacturonic acid, mannuronic acid, gluconic acid, galactonic acid, and mannonic acid. Examples of the amino sugar include glucosamine, mannosamine, and galactosamine.

Preferably, examples of the hexose which may be used in the present invention include naturally-occurring D-glucose, D-mannose, D-galactose, D-glucosamine, D-galactosamine, D-guluronic acid, D-glucuronic acid, D-galacturonic acid, D-mannuronic acid, and L-fucose. More preferably, examples of the hexose which may be used in the present invention include D-glucose, L-fucose, D-glucuronic acid, D-mannose, D-mannuronic acid, and D-guluronic acid.

Examples of the derivative include salt, ester, ether, and amide.

Examples of the salt include an acid addition salt and a base addition salt. Examples of the acid addition salt include a salt derived from an inorganic acid such as hydrochloric acid, hydrobromic acid, hydroiodic acid, sulfuric acid, sulfamic acid, phosphoric acid, and nitric acid, and a salt derived from an organic acid such as p-toluenesulfonic acid, salicylic acid, methanesulfonic acid, oxalic acid, succinic acid, citric acid, malic acid, lactic acid, fumaric acid, and trifluoroacetic acid. Examples of the base addition salt include a salt derived from ammonium, potassium, sodium, or calcium, and for example, a salt derived from a quaternary ammonium hydroxide such as tetramethylammonium hydroxide.

Examples of the ester include a fatty acid ester such as methylester, ethylester, propylester, isopropylester, butylester, isobutylester, t-butylester, pentylester, 1-cyclopropylethylester, and propylene glycol ester, an organic acid ester such as acetic acid ester, sulfonic acid ester, phosphoric acid ester, and malic acid ester, and an inorganic acid ester such as sulfate ester.

Examples of the ether include a lower alkyl ether (e.g., C1-6 alkyl ether) such as methylether and ethylether.

Examples of the amide include a lower alkyl amide (e.g., C1-6 alkyl amide) such as methylamide and ethylamide.

The weight-average molecular weight of the polysaccharide which may be used in the present invention can be measured by a method conventionally used in measuring the weight-average molecular weight of polysaccharide. For example, gel permeation chromatography by HPLC can measure the weight-average molecular weight of polysaccharide. The column used in the measurement may be appropriately selected depending on polysaccharide measured and may be a commercially available column for molecular weight measurement. The method for measuring the weight-average molecular weight is not limited and may be a conventional method.

In one embodiment, the polysaccharide which may be used in the present invention may be a polysaccharide comprising a compound or a derivative thereof, wherein one or more types of sugars represented by Formula (1):

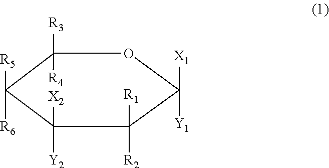

wherein,

### R₁ is H, OH, OSO₃ H, or OZ₁;

Z₁ is a group of the following Formula (2)

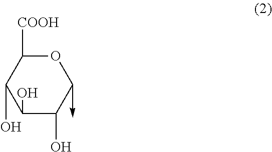

or the following Formula (3),

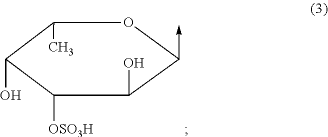

### R₂ is H or OH;

### R₃ is H, CH₃, or CH₂ OH;

### R₄ is H or CH₃;

### R₅ is H;

### R₆ is OH or OSO₃ H;

one of X₁ and Y; is OH and the other is H;
one of X₂ and Y₂ is OH and the other is H,
are linked through at least one binding mode selected from the group consisting of β1,3 linkage, α1,3 linkage, β1,4 linkage, α1,4 linkage, β1,6 linkage, and α1,6 linkage to form the compound (hereinafter may also be referred to as a polysaccharide constructed from sugars of Formula (1)). The polysaccharide constructed from sugars of Formula (1) may have a straight-chain structure or have a side chain.

In one embodiment, the polysaccharide which may be used in the present invention may be a polysaccharide comprising a compound or a derivative thereof, wherein one or more types of sugars of Formula (4):

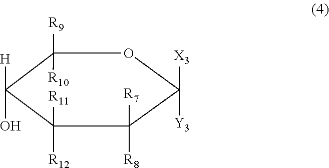

wherein,

### R₇ is H or OH;

### R₈ is H or OH;

### R₉ is H, COOH, CH₂ OH, COOCH₂CH(OH)CH₃, or COOCH(CH₃)CH₂ OH;

### R₁₀ is H, COOH, COOCH₂ CH(OH)CH₃, or COOCH(CH₃)CH₂ OH;

### R₁₁ is OH or OZ₂;

Z₂ is a group of the following Formula (5)

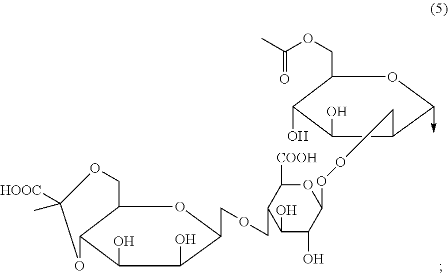

### R₁₂ is H;

one of X₃ and Y₃ is OH and the other is H
are linked through at least one binding mode selected from the group consisting of β1,3 linkage, α1,3 linkage, β1,4 linkage, α1,4 linkage, β1,6 linkage, and α1,6 linkage to form the compound (hereinafter may also be referred to as a polysaccharide constructed from sugars of Formula (4)). The polysaccharide constructed from sugars of Formula (4) may have a straight-chain structure or have a side chain.

In one embodiment, the polysaccharide which may be used in the present invention may be a polymer of Formula (6):

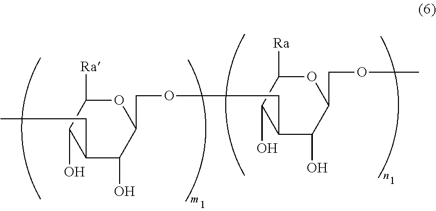

wherein,

### Ra is CH₂ OH;

### Ra′ is CH₂ OH or CH₂ OZ₃,

Z₃ is a group of the following Formula (7)

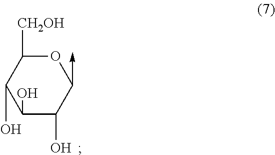

m₁ and n₁ are each an integer selected so that the weight-average molecular weight of the polymer has 10,000 to 10,000,000,
or a derivative thereof (hereinafter may also be referred to as a polymer of Formula (6));
a polymer of Formula (8):

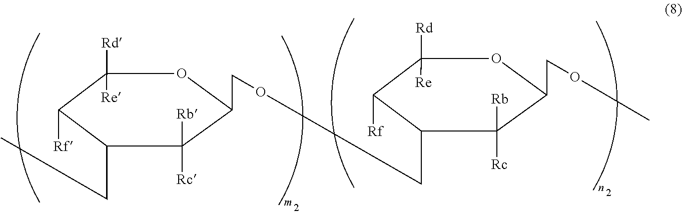

wherein,

### Rb is H, OH, or OSO₃ H;

### Rb′ is OZ₄;

Z₄ is a group of the following Formula (9)

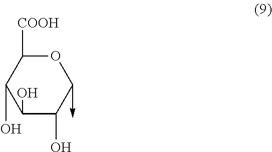

or the following Formula (10),

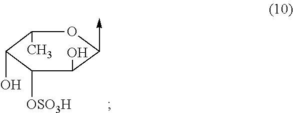

Rc and Rc′ are each independently H or OH;
Rd and Rd′ are each independently H or CH₃;
Re and Re′ are each independently H or CH₃;
Rf or Rf′ is each independently OH or OSO₃ H;
m₂ and n₂ are each an integer selected so that the weight-average molecular weight of the polymer has 1,000 to 10,000,000,
or a derivative thereof (hereinafter may also be referred to as a polymer of Formula (8));
a polymer of Formula (11):

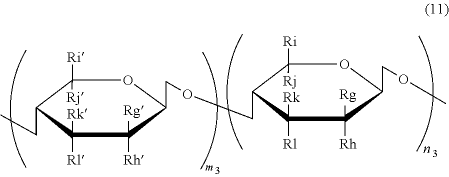

wherein,
Rg and Rg′ are each independently H or OH;
Rh and Rh′ are each independently H or OH;

### Ri is COOH, CH₂ OH, COOCH₂CH(OH)CH₃, or COOCH(CH₃)CH₂ OH;

### Ri′ is H or CH₂ OH;

### Rj is H;

### Rj′ is H, COOH, COOCH₂ CH(OH)CH₃, or COOCH(CH₃)CH₂ OH;

### Rk is OH or OZ₅;

Z₅ is a group of the following Formula (12)

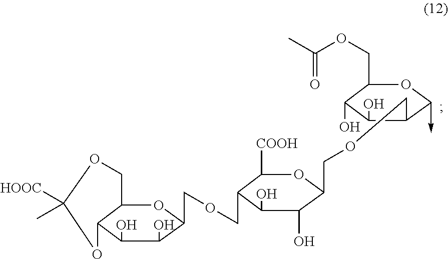

### Rk′ is OH;

Rl and Rl′ are each independently H;
m₃ and n₃ are each an integer selected so that the weight-average molecular weight of the polymer has 10,000 to 50,000,000,
or a derivative thereof (hereinafter may also be referred to as a polymer of Formula (11));
a polymer of Formula (13):

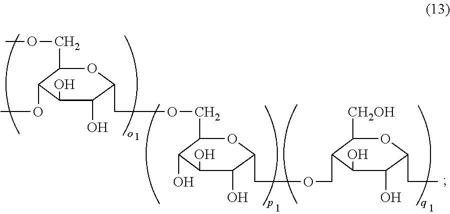

wherein,
o₁, p₁, and q₁ are each an integer selected so that the weight-average molecular weight of the polymer has 1,000 to 10,000,000,
or a derivative thereof (hereinafter may also be referred to as a polymer of Formula (13));
a polymer of Formula (14):

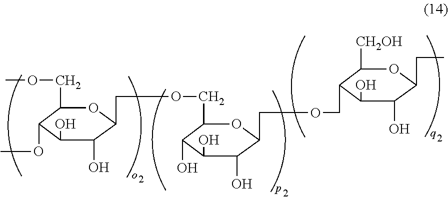

wherein,
o₂, p₂, and q₂ are each an integer selected so that the weight-average molecular weight of the polymer has 1,000 to 10,000,000,
or a derivative thereof (hereinafter may also be referred to as a polymer of Formula (14)); or
a polymer of Formula (15):

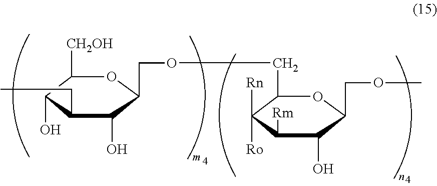

wherein,

### Rm is OH or OSO₃ H;

Rn is H or OH, and when Rn is OH, the hydrogen atom of said OH may be optionally substituted with Z₆, and said Z₆ is a group of the following Formula (16):

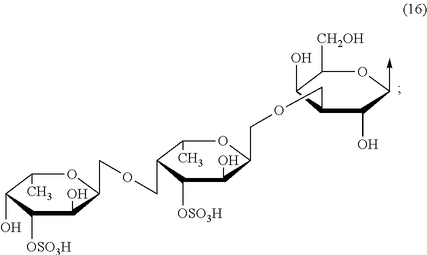

### Ro is H or OH;

m₄ and n₄ are each an integer selected so that the weight-average molecular weight of the polymer has 1,000 to 300,000,
or a derivative thereof (hereinafter may also be referred to as a polymer of Formula (15)).

In the polymer of Formula (6), the ratio of m₁:n₁ is not limited, and may be any ratio of m₁:n₁ ranging from 100:0 to 0:100. Also, m₁ and n₁ may be each an integer selected so that the weight-average molecular weight of the polymer of Formula (6) has, for example, 10,000 to 2,000,000 or 20,000 to 1,300,000. The polymer of Formula (6) may be a homopolymer or a copolymer, and for example, a block copolymer or a random copolymer.

In the polymer of Formula (8), the ratio of m₂:n₂ is not limited, and may be any ratio of m₂:n₂ ranging from 100:0 to 0:100. Also, m₂ and n₂ may be each an integer selected so that the weight-average molecular weight of the polymer of Formula (8) has, for example, 1,000 to 800,000 or 20,000 to 400,000. The polymer of Formula (8) may be a homopolymer or a copolymer, and for example, a block copolymer or a random copolymer.

In the polymer of Formula (11), the ratio of m₃:n₃ is not limited, and may be any ratio of m₃:n₃ ranging from 100:0 to 0:100. Also, m₃ and n₃ may be each an integer selected so that the weight-average molecular weight of the polymer of Formula (11) has, for example, 10,000 to 1,300,000 or 13,000,000 to 50,000,000. The polymer of Formula (11) may be a homopolymer or a copolymer, and for example, a block copolymer or a random copolymer.

In the polymer of Formula (13), the ratio of o₁:p₁:q₁ is 100:0:0/0:100:0/0:0:100. For example, when o₁ is 0, then p₁:q₁ may be 1:2, or when q₁ is 0, then o₁:p₁ may be 100:0 to 0:100. Also, o₁, p₁, and q₁ may be each an integer selected so that the weight-average molecular weight of the polymer of Formula (13) has, for example, 6,000 to 1,400,000 or 50,000 to 1,300,000. The polymer of Formula (13) may be a homopolymer or a copolymer, and for example, a block copolymer or a random copolymer.

In the polymer of Formula (14), o₂:p₂:q₂ is 100:0:0/0:100:0/0:0:100. For example, when o₂ is 0, then p₂:q₂ may be 1:2, or when q₂ is 0, then o₂:p₂ may be 100:0 to 0:100. Also, o₂, p₂, and q₂ may be each an integer selected so that the weight-average molecular weight of the polymer of Formula (13) has, for example, 6,000 to 1,400,000 or 50,000 to 1,300,000. The polymer of Formula (14) may be a homopolymer or a copolymer, and for example, a block copolymer or a random copolymer.

In the polymer of Formula (15), the ratio of m₄:n₄ is not limited, and may be any ratio of m₄:n₄ ranging from 100:0 to 0:100. Also, m₄ and n₄ may be each an integer selected so that the weight-average molecular weight of the polymer of Formula (15) has, for example, 1,000 to 300,000, 1,000 to 200,000, or 20,000 to 100,000. The polymer of Formula (15) may be a homopolymer or a copolymer, and for example, a block copolymer or a random copolymer.

The polysaccharide constructed from sugars of the above Formula (1) or Formula (4), or the polysaccharide of Formula (6), Formula (8), Formula (11), Formula (13), Formula (14), or Formula (15) may be a main chain, and may further contain a side chain containing a hexose residue linked through at least one binding mode selected from the group consisting of β1,6 linkage, α1,2 linkage, β1,3 linkage, α1,3 linkage, and α1,6 linkage. The number of sugar residue in the side chain is not limited, and may be 1, 2, 3, 4, 5, or more.

The polysaccharide constructed from sugars of the above Formula (1) or Formula (4), or the polysaccharide of Formula (6), Formula (8), Formula (11), Formula (13), Formula (14), or Formula (15) may be esterified at the hydroxyl group or carboxyl group. For example, all or a part of the hydroxyl groups in the polysaccharide may be modified with a sulfate group.

Examples of the polysaccharide which may be used in the present invention include amylose, amylopectin, glycogen, cellulose, starch, chitin, chitosan, agarose, carrageenan, hyaluronic acid, chondroitin 4-sulfate, chondroitin 6-sulfate, dermatan sulfate, keratan sulfate, heparan sulfate, heparin, xyloglucan, glucomannan, galactomannan, laminaran, xylan, pectin, lentinan, maltodextrin, curdlan, guar gum, β-1,3-glucan, fucoidan, alginic acid, alginic acid ester, pullulan, xanthan gum, dextran, and laminaran. More specifically, examples of the polysaccharide which may be used in the present invention include the following polysaccharides.

(a) β-1,3-Glucan

The β-1,3-glucan which may be used in the present invention may be any glucan containing β1,3 linkage, and may be a glucan constructed from glucose residues linked through β1,3 linkage, or a glucan containing a main chain constructed from glucose residues linked through β1,3 linkage and further containing a side chain, or a mixture thereof. The β-1,3-glucan may be prepared from a natural product such as Aureobasidium sp. microorganism, a baker's yeast such as S. cerevisiae, a basidiomycete such as Lentinus edodes, Schizophyllum Commune, and Coriolus versicolor, euglenoid, and gramineous cereal. The β-1,3-glucan which may be used in the present invention may be derived from any source.

Examples of the β-1,3-glucan which may be used in the present invention include, but are not limited to, a β-1,3-1,6-glucan derived from Aureobasidium pullulans such as one available as Aqua β (registered trademark) manufactured by DAISO Co., Ltd., a β-1,3-1,6-glucan derived from baker's yeast such as one available as Wellmune (registered trademark) manufactured by BIOTHERA Inc., and paramylon, i.e., a straight chain β1,3-glucan derived from euglenoid. An example of the β-1,3-glucan has the following Formula (17).

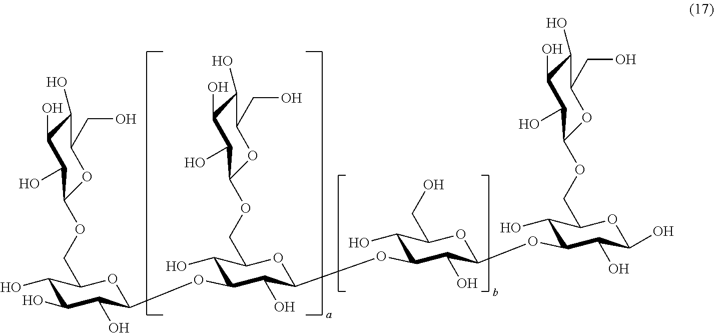

In the above formula, a and b are each any integer. For example, a and b may be selected so that the weight-average molecular weight of the polymer has 10,000 to 10,000,000 such as 20,000 to 300,000 or 500,000 to 2,500,000, preferably 10,000 to 2,000,000, more preferably 20,000 to 1,300,000. The branching degree of β-1,3 linkage/β-1,6 linkage, i.e., the linkage ratio of β1,6 side chain to β1,3 main chain may be an integer about 30 to 100% for a glucan derived from Aureobasidium, an integer about 33% for a glucan derived from a mushroom such as Schizophyllum commune, an integer about 0% for a glucan derived from euglenoid.

The β-1,3-glucan which may be used in the present invention may be sulfated at some hydroxyl groups of glucose residues. For example, some hydroxyl groups of the β-1,3-glucan of Formula (17) may be converted into OSO₃ H. The β-1,3-glucan which may be used in the present invention may be in the form of a salt such as sodium, potassium, calcium, and ammonium salt.

(b) Fucoidan

The fucoidan which may be used in the present invention is a compound wherein ten to hundreds of thousands of L-fucose residues are linked with each other, and may be a fucoidan constructed from linked L-fucose residues, or a fucoidan containing a main chain constructed from linked L-fucose residues and containing a side chain, or a mixture thereof. The fucoidan may be derived from any source. For example, a fucoidan derived from a brown algae such as Mozuku (Nemacystus decipiens), mekabu seaweed, and tangle weed may be used in the present invention. Examples of such fucoidan include a fucoidan obtainable from natural Mozuku (Nemacystus decipiens) made in Kingdom of Tonga such as one available as Meito-fucoidan manufactured by Kyodo Milk Industry Co., Ltd. and a fucoidan derived from Kjellmaniella crassifolia such as one available as Fucoidan from Kjellmaniella crassifolia manufactured by TAKARA BIO INC. The fucoidan contains sulfate groups in the chemical structure. For example, the fucoidan may contain 2 to 12, 4 to 12, 5 to 10, or 8 to 10 sulfate groups per 5 sugar residues. The fucoidan may contain 10 to 40% by weight, 15 to 30% by weight, or 20 to 30% by weight of sulfate groups.

An example of the fucoidan has the following Formula (18).

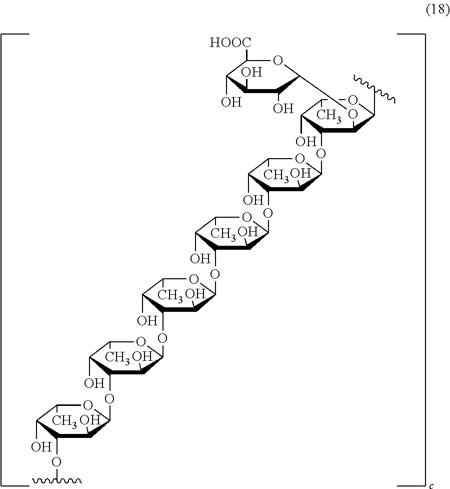

In the above formula, c is any integer, and may be selected no that the weight-average molecular weight of the polymer has 5,000 or more such as a weight-average molecular weight of 1,000 to 300,000, preferably 1,000 to 200,000, more preferably 20,000 to 100,000.

The fucoidan derived from Kjellmaniella crassifolia is a mixture of U-fucoidan of the following Formula (19), G-fucoidan of the following Formula (20), and F-fucoidan of the following Formula (21).

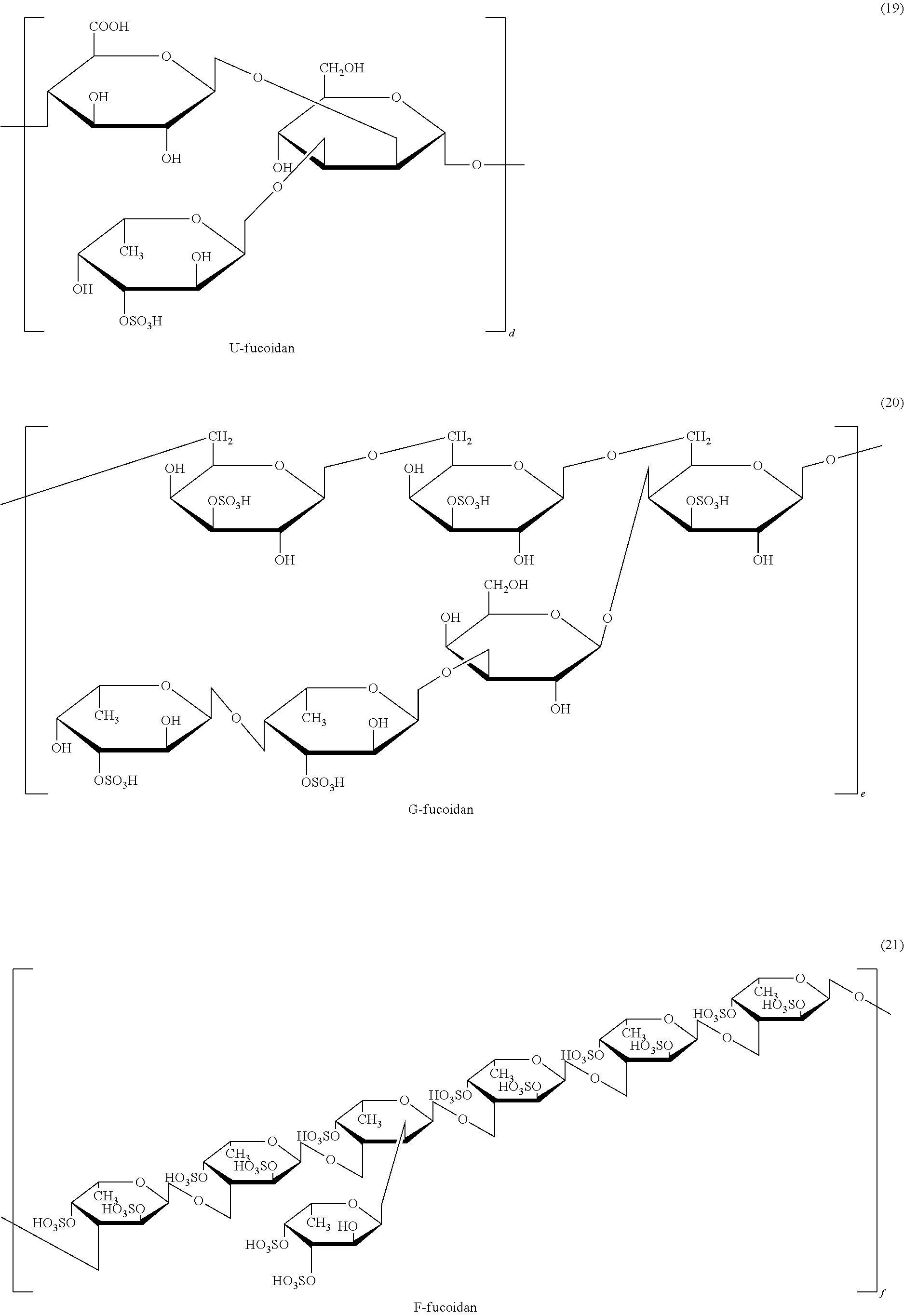

In the above formulas, d, e, and f are each any integer, and may be selected so that the weight-average molecular weight of the mixture has about 200,000 such as a weight-average molecular weight of 1,000 to 10,000,000, preferably 20,000 to 800,000, more preferably 50,000 to 400,000.

The fucoidan which may be used in the present invention may be in the form of a salt such as sodium, potassium, calcium, and ammonium salt.

(c) Alginic Acid and Alginic Acid Derivative

The alginic acid which may be used in the present invention is a straight polymer constructed from two types of blocks of β-D-mannuronic acid and the C-5 epimer thereof, i.e., a-L-guluronic acid linked through α1,4 linkage. The alginic acid which may be used in the present invention may be prepared from a brown algae, but may be derived from any source. As the alginic acid, an alginic acid salt such as sodium alginate, potassium alginate, ammonium alginate, and calcium alginate may be used in the present invention, and an alginic acid ester such as alginic acid propylene glycol ester may be used in the present invention. Sodium alginate and alginic acid propylene glycol ester are available, for example, from Wako Pure Chemical Industries, Ltd. or KIMICA Corporation.

The alginic acid has the following Formula (22).

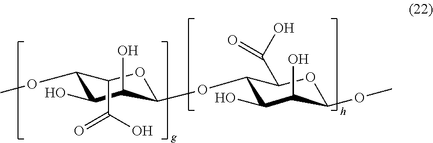

In the above formula, g and h are each any integer, and for example, may be selected so that the weight-average molecular weight of the polymer has about 240,000 or 380,000 such as the weight-average molecular weight of 10,000 to 600,000, preferably 10,000 to 500,000, more preferably 20,000 to 450,000.

The weight-average molecular weight of alginic acid ester may be 10,000 to 600,000, preferably 10,000 to 400,000, more preferably 20,000 to 200,000.

(d) Pullulan

The pullulan which may be used in the present invention has a structure, wherein three glucose molecules are linked through α1,4 linkage to form a maltotriose molecule and maltotriose molecules are linked through α1,6 linkage to form a pullulan molecule (see the following Formula (23), wherein i is any integer). The pullulan is produced by Aureobasidium pullulans, for example, from sucrose or starch hydrolysate.

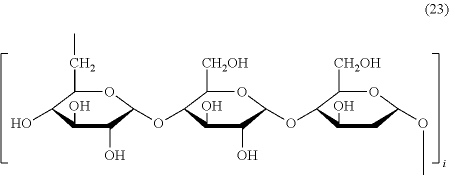

Examples of the pullulan which may be used in the present invention include Shodex standard P-1300 (weight-average molecular weight: about 1,330,000; the value in catalog) manufactured by SHOWA DENKO K.K. The weight-average molecular weight of pullulan which may be used in the present invention may be preferably 100,000 to 14,000,00, more preferably 500,000 to 1,300,000.

The pullulan which may be used in the present invention may be in the form of a salt such as sodium, potassium, calcium, and ammonium salt.

(e) Xanthan Gum

The xanthan gum which may be used in the present invention is a compound comprising the repeating unit of two glucose molecules, two mannose molecules, and glucuronic acid, and a derivative thereof. For example, the xanthan gum may be SATIAXANE (registered trademark) CX90 manufactured by UNITEC FOODS CO. LTD., and may be a compound containing a main chain constructed from glucose residues linked through β1,4 linkage and a side chain consisting of a derivative of two mannose molecules and a glucuronic acid molecule as shown in the following Formula (24).

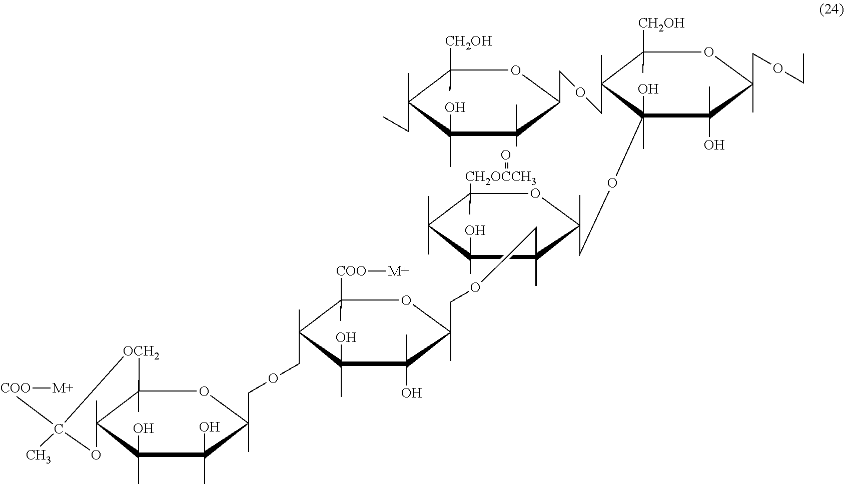

In the above formula, M+ may be, for example, sodium, potassium, or calcium. The xanthan gum which may be used in the present invention may be in the form of a salt such as sodium, potassium, calcium, and ammonium salt. The weight-average molecular weight of xanthan gum may be, for example, about 2,000,000 or 13,000,000 to 50,000,000.

(f) Dextran

The dextran which may be used in the present invention is a compound comprising the repeating unit of glucose molecules and a derivative thereof. For example, a dextran produced by Leuconostoc mesenteroides may be used, and may be a compound containing a main chain constructed from glucose residues linked through α1,6 linkage and a side chain consisting of a derivative of a glucose molecule as shown in the following Formula (25).

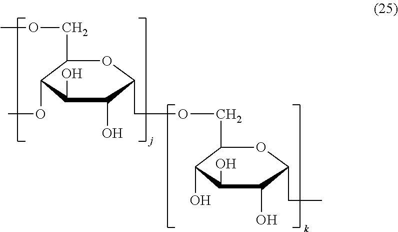

In the above formula, j and k are each any integer, and for example, may be selected so that the weight-average molecular weight of the polymer becomes 1,000 to 10,000,000, preferably 6,000 to 800,000, more preferably 50,000 to 400,000.

(g) Laminaran

The laminaran which may be used in the present invention is a compound comprising the repeating unit of glucose molecules, and a derivative thereof. The laminaran may be derived from any source. For example, a laminaran derived from a brown algae such as Mozuku (Nemacystus decipiens), mekabu seaweed, and tangle weed may be used, and may be a compound consisting of a main chain constructed from glucose residues randomly linked through β1,6 linkage and β1,3 linkage as shown in the following Formula (26).

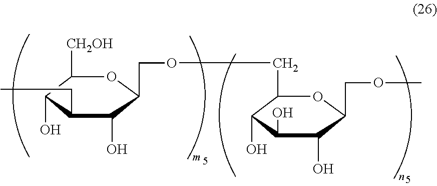

In the above formula, m₅ and n₅ are each an integer selected so that the weight-average molecular weight of the polymer has, for example, 1,000 to 300,000, preferably 1,000 to 200,000, more preferably 20,000 to 100,000.

Further, the “polysaccharide which may be used in the present invention” as described above may be a polysaccharide which is degraded into a lower-molecular compound by a known method. Examples of the method for degradation into a lower-molecular compound include any known polysaccharide hydrolysis reactions. For example, a water-soluble polysaccharide is known to hydrolyze in the presence of an acid under pressure and heating, and the polysaccharide may be degraded into a lower-molecular compound by the hydrolysis. Further, a polysaccharide may be degraded into a lower-molecular compound using a hydrolysis reaction by an enzyme. In addition, a polysaccharide may be degraded into a lower-molecular compound by a physical treatment such as sonication.

The “polysaccharide which may be used in the present invention” as described above may be used in the enhanced production of a useful substance in cells. Preferably, provided is the use of the “polysaccharide which may be used in the present invention” for the enhanced production of a useful substance using cells in vitro. Regardless of the mechanism, the enhanced production may increase the production of a useful substance within a period of time. For example, the production of a useful substance may be increased by promoting the cell growth, or by increasing the expression of a useful substance in an individual cell, or both. Thus, the “polysaccharide which may be used in the present invention” as described above may be used as a cell growth promoter, or as an expression promoter of a useful substance in a cell, or both.

The “polysaccharide which may be used in the present invention” as described above may be used in the enhanced production of a useful substance by adding said polysaccharide to a medium for cell culture. In a preferable embodiment, the “polysaccharide which may be used in the present invention” as described above may be added to a medium for cell culture under a serum-free condition or a condition with reduced concentration of serum such as 5, 3, or 1% FBS-containing condition to promote the production of a useful substance. Thus, the “polysaccharide which may be used in the present invention” as described above may be used alone as a medium additive, or may be used as a medium additive for cell culture together with other substances for enhanced production of a useful substance such as a protein (e.g., transferrin) and/or a low-molecular compound (e.g., glucose, phosphate, and selenious acid).

The composition provided by the present invention such as a composition for enhanced production of a useful substance and a medium additive for cell culture may contain the “polysaccharide which may be used in the present invention” in an amount such as 0.0001 to 50% by weight, 0.0001 to 10% by weight, preferably 0.0005 to 1% by weight, more preferably 0.001 to 0.1% by weight. The composition may be liquid or solid at room temperature. For example, the “polysaccharide which may be used in the present invention” may be dissolved in a buffer such as PBS at the above amount to prepare the composition of the present invention.

Examples of the medium to which the “polysaccharide which may be used in the present invention” as described above is added include EMEM, αMEM, DMEM, Ham's medium, RPMI1640, Fisher's medium, and a mixture thereof. The “polysaccharide which may be used in the present invention” as described above may be added to these basic media to prepare a serum-free medium or a medium with reduced concentration of serum such as a 5, 3, or 1% FBS-containing medium useful in the manufacture of a useful substance. Also, the “polysaccharide which may be used in the present invention” may be added to a serum-free medium, a protein-free medium, or a Chemically defined medium containing a growth factor or a serum replacement and used under a serum-free condition. Examples of these media include, but are not limited to, X-CELL 302, EX-CELL 325-PF, and EX-CELL CD CHO manufactured by SAFC Biosciences, Inc., SFM II, CHO-III-PFM, and CD CHO manufactured by Life Technologies Corporation, and IS-CHO CD and BalanCD Growth A Medium manufactured by Irvine Scientific. Further, the “polysaccharide which may be used in the present invention” may be added to a mixed medium in which two or more of said serum-free medium, protein-free medium, or Chemically defined medium are mixed at any ratio. The amount of the “polysaccharide which may be used in the present invention” in the medium is not limited, and may be, for example 1 to 10,000 μg/ml, preferably 1 to 5,000 μg/ml, more preferably 1 to 2,000 μg/ml. For example, those skilled in the art, without altering the concentration of each ingredient other than water in the medium to which the “polysaccharide which may be used in the present invention” as described above is added, could add the “polysaccharide which may be used in the present invention” to the medium to prepare a medium containing the “polysaccharide which may be used in the present invention” at 1 to 10,000 μg/ml, preferably 1 to 5,000 μg/ml, more preferably 1 to 2,000 μg/ml. In addition to the “polysaccharide which may be used in the present invention”, the medium comprising the “polysaccharide which may be used in the present invention” may comprise a substance selected from, for example, an inorganic salt such as sodium salt, potassium salt, and calcium salt, a carbohydrate such as a sugar, e.g., glucose, and an amino acid such as an essential amino acid, a vitamin such as riboflavin and thiamine, a fatty acid or lipid such as a steroid, e.g., cholesterol, a protein or peptide such as albumin and transferrin, a trace element such as zinc, copper, and selenium, and a combination thereof. In one embodiment, in addition to the “polysaccharide which may be used in the present invention”, a liquid medium comprising the “polysaccharide which may be used in the present invention” contains 0.1 to 4.5 g/L of glucose, 0.1 to 0.5 g/L of CaCl₂, 1 to 10 g/L of NaCl, 0.001 to 0.3 g/L of L-arginine.HCl, 0.001 to 0.3 g/L of L-cysteine.2HCl, 0.001 to 0.3 g/L of L-histidine.HCl.H₂ 0, 0.001 to 0.3 g/L of L-isoleucine, 0.001 to 0.3 g/L of L-leucine, 0.001 to 0.3 g/L of L-lysine.HCl, 0.001 to 0.3 g/L of L-methionine, 0.001 to 0.3 g/L of L-phenylalanine, 0.001 to 0.3 g/L of L-threonine, 0.001 to 0.3 g/L of L-tryptophan, 0.001 to 0.3 g/L of L-tyrosine.2Na.2H₂O, and 0.001 to 0.3 g/L of L-valine. In one embodiment, the medium may be a powder medium which is turned into a medium having the above constitution when dissolved in water. Said medium containing the “polysaccharide which may be used in the present invention” as described above may be used in the manufacture of a useful substance.

Specifically, a useful substance may be prepared by a method comprising culturing cells which produce a useful substance in a medium comprising the “polysaccharide which may be used in the present invention” as described above, and isolating the produced useful substance from the cells. Said medium may be a serum-free medium or a medium with a reduced concentration of serum such as a 5, 3, or 1% FBS-containing medium. For example, when an antibody is prepared as a useful substance, the antibody may be prepared by a method comprising culturing antibody-producing cells in a medium comprising the “polysaccharide which may be used in the present invention” as described above, and purifying the antibody. The purification steps of the antibody include, for example, protein A affinity column chromatography, viral inactivation by a low pH treatment, other chromatography such as cation exchange chromatography and anion exchange chromatography, filtration by a virus removal filter, concentration, and the final filtration.

The useful substance as used herein is not limited, and may be a substance useful in a medicine, an agrichemical, a food, and other chemical industry. Preferably, examples of the useful substance include a bioactive protein or peptide such as an antibody, an enzyme such as urokinase, a hormone such as insulin, and a cytokine such as interferon, interleukin, erythropoietin, G-CSF, and GM-CSF. Examples of the antibody, include a mouse monoclonal antibody, a humanized monoclonal antibody, or a human monoclonal antibody. The class of immunoglobulin is not limited, and for example, may be IgG such as IgG1 and IgG2. The useful substance may be a recombinant protein which is an expression product of a foreign gene.

The cells as used herein are not limited and may be cells usable in the production of a useful substance such as a recombinant protein. Examples of the cells include CHO cells, BHK cells, HepG2 cells, rodent myeloma cells such as mouse myeloma cells, for example SP2/O cells and NSO cells, hybridomas, insect cells, and transformed cells of these cells wherein a foreign gene is transfected. When an antibody is produced as the useful substance, antibody-producing cells such as hybridomas obtainable by fusion of cells such as CHO cells, SP2/O cells, and NSO cells may be used.

Hereinafter, the present invention is further illustrated by Examples, but the present invention is not limited to them.

### Examples

A CHO cell line (ATCC CRL-12445) transfected with an IgG gene which secretes and produces an IgG antibody was purchased from ATCC and used. Said CHO cell line was cultured in 10% FBS-containing DMEM and then harvested, suspended in 1% FBS-containing DMEM, and seeded into a 12 well multiplate at 4×10⁴ cells/well. The following test polysaccharide samples were dissolved in sterile ultrapure water, and added to each well at the final concentration of 40, 200, 1,000 μg/ml. Meanwhile, sterile ultrapure water was added as a control at the equal amount to the samples. After cultured for 3 days, each IgG concentration was measured. The resulting IgG concentration of each cell supernatant in the well to which each test polysaccharide sample was added is shown as a relative value in Tables 1-1 to 1-4, wherein the IgG concentration of the cell supernatant in the control well, i.e., the IgG concentration in the cell supernatant of the well to which only sterile ultrapure water was added, was taken as 100. The following test samples all increased the IgG production.

Test Sample:

β-1,3-1,6-Glucan derived from Aureobasidium pullulans manufactured by DAISO Co., Ltd.; molecular weight: 800,000 and 80,000.

The β-1,3-1,6-glucan having the molecular weight of 80,000 was prepared by sonication (at 4° C., for 4 hours, using ultrasonic homogenizer (SONIFIER 250) manufactured by BRANSON) of the β-1,3-1,6-glucan having the molecular weight of 800,000.

β-1,3-1,6-Glucan derived from baker's yeast (manufactured by BIOTHERA Inc.);

Paramylon (manufactured by Wako Pure Chemical Industries, Ltd.);

Fucoidan derived from Mozuku (Nemacystus decipiens) (manufactured by Kyodo Milk Industry Co., Ltd.);

Fucoidan derived from tangle weed (manufactured by TAKARA BIO INC.);

Alginic acid propylene glycol ester (manufactured by KIMICA Corporation);

Sodium alginate (manufactured by Wako Pure Chemical Industries, Ltd.);

Pullulan (Shodex standard P-1300 manufactured by SHOWA DENKO K.K.);

Xanthan gum (SATIAXANE CX90 manufactured by UNITEC FOODS CO. LTD.);

Dextran (manufactured by Wako Pure Chemical Industries, Ltd.)

Laminaran (manufactured by Tokyo Chemical Industry Co., Ltd.)

Method for Measuring Weight-Average Molecular Weight of Test Sample (Organic Solvent System)

The weight-average molecular weight of each sample of β-1,3-1,6-glucan derived from Aureobasidium pullulans, β-1,3-1,6-glucan derived from baker's yeast, paramylon, and dextran used in Examples was measured according to the following method. The obtained weight-average molecular weight is shown in Tables 1-1 to 1-4.

Device: High performance liquid chromatography (manufactured by Nihon Waters K.K.)
Column: Shodex GPC KD-806M (column size: 8×300 mm) (manufactured by SHOWA DENKO K.K.)
Column temperature: 50° C.
Mobile phase: 10 mM LiBr/DMSO solution
Flow rate: 0.7 ml/min

### Inflow: 100 μl

Sample concentration: 0.10% (1 mg/ml)
Detector: 2414 differential refractive index detector (RI detector) (manufactured by Nihon Waters K.K.)
Pretreatment: 0.45 μm membrane filter filtration (Minisart RC 4)
Molecular weight marker: pullulan (weight-average molecular weight; 1,330,000, 788,000, 404,000, 212,000, 112,000, 47,300, 22,800, 11,800) (manufactured by SHOWA DENKO K.K.)

Method for Measuring Weight-Average Molecular Weight of Test Sample (Aqueous System)

The weight-average molecular weight of each sample of fucoidan derived from Mozuku (Nemacystus decipiens), fucoidan derived from tangle weed, alginic acid propylene glycol ester, sodium alginate, pullulan, xanthan gum, and laminaran used in Examples was measured according to the following method. The obtained weight-average molecular weight is shown in Tables 1-1 to 1-4. A TOSHO guard column was tandemly connected with two TSK-GEL GMPW_{X L} Columns to measure the weight-average molecular weight.

Device: High performance liquid chromatography (manufactured by Nihon Waters K.K.)
Column: two TSK-GEL GMPW_{X L} Columns (column size: 7.5 mm I.D×30 cm) (manufactured by TOSOH CORPORATION), TOSHO guard column
Column temperature: 40° C.
Mobile phase: 200 mM K₂ HPO₄ aqueous solution
Flow rate: 1.0 ml/min

### Inflow: 100 μl

Sample concentration: 0.20% (2 mg/ml)
Detector: 2414 differential refractive index detector (RI detector) (manufactured by Nihon Waters K.K.)
Pretreatment: 0.45 μm membrane filter filtration (Minisart RC 4)
Molecular weight marker: pullulan (weight-average molecular weight; 1,330,000, 788,000, 212,000, 47,300, 22,800, 11,800, 5,900) (manufactured by SHOWA DENKO K.K.)

Method for Measuring IgG Production

Human IgG ELISA measuring kit (Human IgG ELISA Quantitation Set, ELISA Starter Accessory Kit) manufactured by Bethyl Laboratories, Inc. was used according to the method described in the attached instruction manual to measure the IgG concentration in the cell culture medium.

TABLE 1-1

| | Polysaccharide name | | |
|---|---|---|---|
| | β-1,3-1,6 | β-1,3-1,6 | β-1,3-1,6 |
| | Glucan | Glucan | Glucan |
| Weight-average | 800,000 | 80,000 | 470,000 |
| molecular weight | | | |
| (Mw) | | | |
| Main component of | Glucose | Glucose | Glucose |
| constituent sugar | | | |
| Binding mode | Main chain: | Main chain: | Main chain: |
| | β1,3 linkage | β1,3 linkage | β1,3 linkage |
| | Side chain: | Side chain: | Side chain: |
| | β1,6 linkage | β1,6 linkage | β1,6 linkage |
| Source | Aureobasidium | Aureobasidium | Baker's yeast |
| | pullulans | pullulans | |
| | IgG production | IgG production | IgG production |
| 40 μg/ml | 104% | 109% | 134% |
| 200 μg/ml | 111% | 140% | 106% |
| 1,000 μg/ml | 160% | 163% | 121% |

TABLE 1-2

| | Polysaccharide name | | |
|---|---|---|---|
| | Paramylon | Fucoidan | Fucoidan |
| Weight-average | 260,000 | 70,000 | 190,000 |
| molecular weight | | | |
| (Mw) | | | |
| Main component | Glucose | Fucose, | Fucose, |
| of constituent | | Glucuronic | Glucuronic |
| sugar | | acid | acid |
| Binding mode | Main chain: | Main chain: | Mixture of |
| | β1,3 linkage | α1,3 linkage | fucoidan |
| | | Side chain: | (U, F, G) |
| | | α1,2 linkage | |
| Source | Euglenoid | Mozuku | Tangle weed |
| | | (Nemacystus | |
| | | decipiens) | |
| | IgG production | IgG production | IgG production |
| 40 μg/ml | 115% | 103% | 105% |
| 200 μg/ml | 148% | 156% | 107% |
| 1,000 μg/ml | 152% | 190% | 142% |

TABLE 1-3

| | Polysaccharide name | | |
|---|---|---|---|
| | Alginic acid | Sodium | |
| | ester | alginate | Pullulan |
| Weight-average | 100,000 | 380,000 | 800,000 |
| molecular weight | | | |
| (Mw) | | | |
| Main component | Mannuronic | Mannuronic | Glucose |
| of constituent | acid, | acid, | |
| sugar | Guluronic acid | Guluronic acid | |
| Binding mode | Main chain: | Main chain: | Main chain: |
| | mixture of | mixture of α1,4 | α1,4 linkage |
| | α1,4 linkage and | linkage and | and α1,6 |
| | β1,4 linkage | β1,4 linkage | linkage |
| Source | Brown algae | Brown algae | Aureobasidium |
| | | | pullulans |
| | IgG production | IgG production | IgG production |
| 40 μg/ml | 105% | 112% | 113% |
| 200 μg/ml | 116% | 109% | 109% |
| 1,000 μg/ml | 143% | 121% | 124% |

TABLE 1-4

| | Polysaccharide name | | |
|---|---|---|---|
| | Xanthan gum | Dextran | Laminaran |
| Weight-average | 800,000 or more | 200,000 | 33,000 |
| molecular weight | | | |
| (Mw) | | | |
| Main component of | Glucose, Mannose, | Glucose | Glucose |
| constituent sugar | Glucuronic acid | | |
| Binding mode | Main chain: β1,4 | Main chain: | Main chain: |
| | linkage | α1,6 linkage | β1,3 linkage |
| | | Side chain: | and β1,6 |
| | | α1,4 linkage | linkage |
| Source | Xanthomonas | Leuconostoc | Eisenia |
| | campestris | mesenteroides | bicyclis |
| | IgG production | IgG production | IgG production |
| 40 μg/ml | 128% | 114% | 100% |
| 200 μg/ml | 123% | 106% | 128% |
| 1,000 μg/ml | 120% | 103% | 167% |

The composition, medium, and process of the present invention may be used in the production of a useful substance using cells.

In one embodiment, the present invention can provide an inexpensive, safe, and excellent composition for enhanced production of a useful substance comprising a polysaccharide as an active ingredient. A medium for cell culture comprising a polysaccharide of the present invention can promote the growth of cells regardless of the form or type of the cells, and increase the production of a useful substance. Thus, in one embodiment, the composition, medium, and process of the present invention can increase the production of a useful substance in cells, and can be applied in various fields such as preparation of a clinical diagnostic agent using an antibody, production of an antibody drug, regenerative medicine, and cell therapy.

1. A composition for enhanced production of a useful substance in cells comprising a polysaccharide, wherein the polysaccharide contains hexose residues linked through a binding mode between carbon at 1-position and carbon at 3-position, carbon at 1-position and carbon at 4-position, or carbon at 1-position and carbon at 6-position.

2. A medium for cell culture comprising a polysaccharide, wherein the polysaccharide contains hexose residues linked through a binding mode between carbon at 1-position and carbon at 3-position, carbon at 1-position and carbon at 4-position, or carbon at 1-position and carbon at 6-position.

3. The composition or medium according to claim 1, wherein the hexose is a sugar containing an aldehyde group.

4. The composition or medium according to claim 1, wherein the hexose is a deoxy sugar, a sugar containing carboxylic acid, or an amino sugar.

5. The composition or medium according to claim 1, wherein the hexose is at least one sugar selected from the group consisting of allose, altrose, glucose, mannose, gulose, idose, galactose, talose, fucose, rhamnose, fuculose, and a derivative thereof.

6. The composition or medium according to claim 1, wherein the hexose residues are linked through at least one binding mode selected from the group consisting of β1,3 linkage, α1,3 linkage, β1,4 linkage, α1,4 linkage, β1,6 linkage, and α1,6 linkage.

7. The composition or medium according to claim 1 comprising a polysaccharide or a derivative thereof, wherein one or more types of sugars represented by Formula (1): 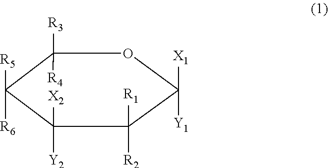
wherein,
R₁ is H, OH, OSO₃ H, or OZ₁;
Z₁ is a group of the following Formula (2) 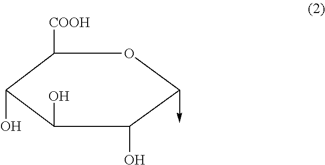
or the following Formula (3), 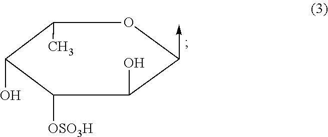
R₂ is H or OH;
R₃ is H, CH₃, or CH₂ OH;
R₄ is H or CH₃;
R₅ is H;
R₆ is OH or OSO₃ H;
one of X₁ and Y₁ is OH and the other is H;
one of X₂ and Y₂ is OH and the other is H,
are linked through at least one binding mode selected from the group consisting of β1,3 linkage, α1,3 linkage, β1,4 linkage, α1,4 linkage, β1,6 linkage, and α1,6 linkage to form the polysaccharide.

8. The composition or medium according to claim 1 comprising a polysaccharide or a derivative thereof, wherein one or more types of sugars represented by Formula (4): 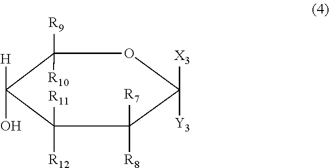
wherein,
R₇ is H or OH;
R₈ is H or OH;
R₉ is H, COOH, CH₂ OH, COOCH₂ CH(OH)CH₃, or COOCH(CH₃)CH₂ OH;
R₁₀ is H, COOH, COOCH₂ CH(OH)CH₃, or COOCH(CH₃)CH₂ OH;
R₁₁ is OH or OZ₂;
Z₂ is a group of the following Formula (5) 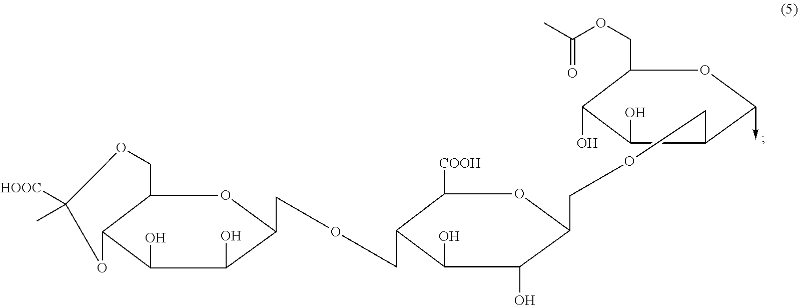
R₁₂ is H;
one of X₃ and Y₃ is OH and the other is H,
are linked through at least one binding mode selected from the group consisting of β1,3 linkage, α1,3 linkage, β1,4 linkage, α1,4 linkage, β1,6 linkage, and α1,6 linkage to form the polysaccharide.

9. The composition or medium according to claim 1, wherein the polysaccharide is a polymer of Formula (6): 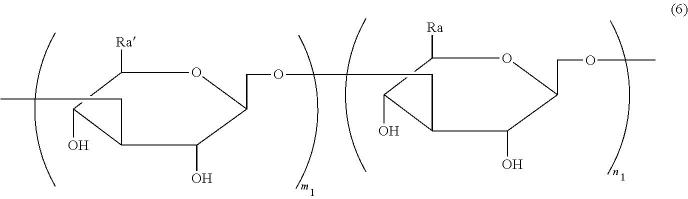
wherein,
Ra is CH₂ OH;
Ra′ is CH₂ OH or CH₂ OZ₃,
Z₃ is a group of the following Formula (7) 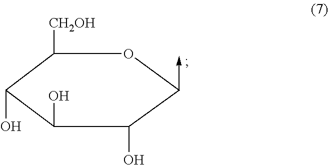
m₁ and n₁ are each an integer selected so that the weight-average molecular weight of the polymer has 10,000 to 10,000,000,
or a derivative thereof;
a polymer of Formula (8): 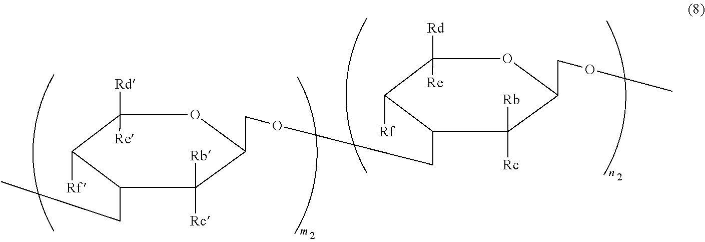
wherein,
Rb is H, OH, or OSO₃ H;
Rb′ is OZ₄;
Z₄ is a group of the following Formula (9) 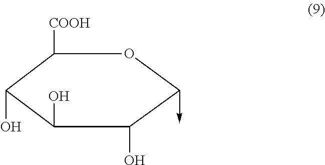
or the following Formula (10), 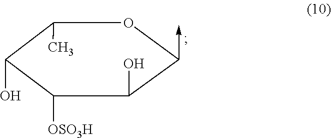
Rc and Rc′ are each independently H or OH;
Rd and Rd′ are each independently H or CH₃;
Re and Re′ are each independently H or CH₃;
Rf or Rf′ is each independently OH or OSO₃ H;
m₂ and n₂ are each an integer selected so that the weight-average molecular weight of the polymer has 1,000 to 10,000,000,
or a derivative thereof;
a polymer of Formula (11): 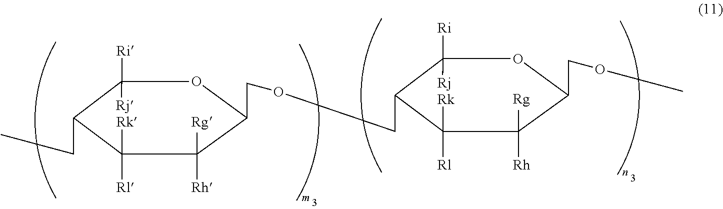
wherein,
Rg and Rg′ are each independently H or OH;
Rh and Rh′ are each independently H or OH;
Ri is COOH, CH₂ OH, COOCH₂ CH(OH)CH₃, or COOCH(CH₃)CH₂ OH;
Ri′ is H or CH₂ OH;
Rj is H;
Rj′ is H, COOH, COOCH₂ CH(OH)CH₃, or COOCH(CH₃)CH₂ OH;
Rk is OH or OZ₅;
Z₅ is a group of the following Formula (12) 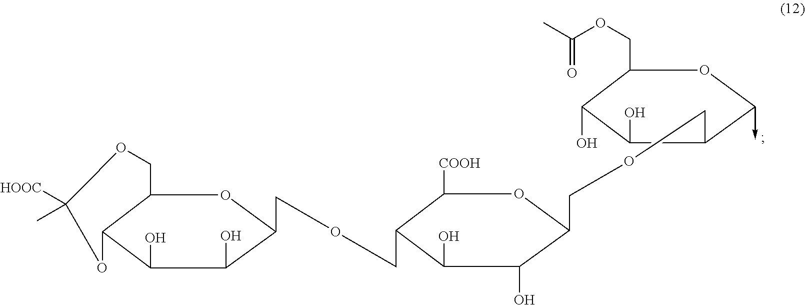
Rk′ is OH;
Rl and Rl′ are each independently H;
m₃ and n₃ are each an integer selected so that the weight-average molecular weight of the polymer has 10,000 to 50,000,000,
or a derivative thereof;
a polymer of Formula (13): 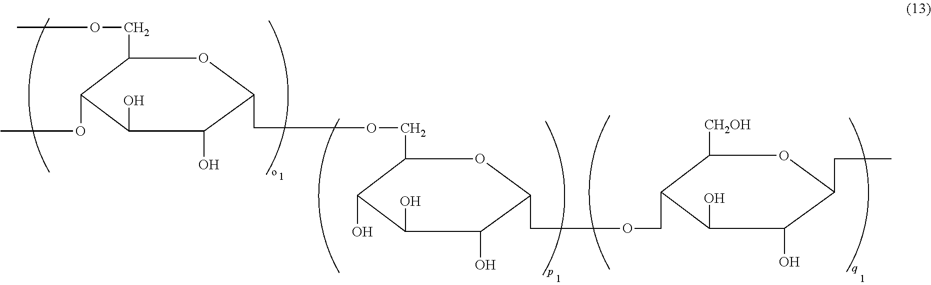
wherein,
o₁, p₁, and q₁ are each an integer selected so that the weight-average molecular weight of the polymer has 1,000 to 10,000,000,
or a derivative thereof;
a polymer of Formula (14): 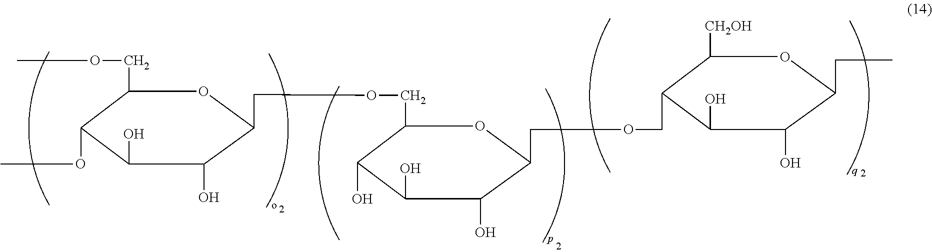
wherein,
o₂, p₂, and q₂ are each an integer selected so that the weight-average molecular weight of the polymer has 1,000 to 10,000,000,
or a derivative thereof; or
a polymer of Formula (15): 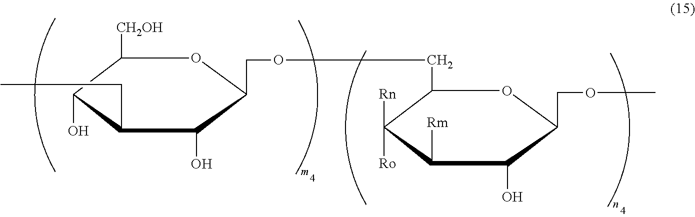
wherein,
Rm is OH or OSO₃ H;
Rn is H or OH, and when Rn is OH, the hydrogen atom of said OH may be optionally substituted with Z₆, and said Z₆ is a group of the following Formula (16): 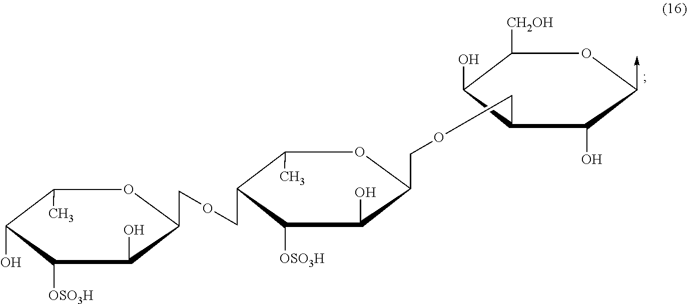
Ro is H or OH;
m₄ and n₄ are each an integer selected so that the weight-average molecular weight of the polymer has 1,000 to 300,000,
or a derivative thereof.

10. The composition or medium according to claim 1, wherein the polysaccharide further contains a hexose residue linked through at least one binding mode selected from the group consisting of β1,6 linkage, α1,2 linkage, β1,3 linkage, α1,3 linkage, and α1,6 linkage.

11. The composition or medium according to claim 1, wherein a part or all of the hydroxyl groups of the polysaccharide are further modified with a sulfate group.

12. The composition or medium according to claim 1, wherein the useful substance is an IgG antibody.

13. The composition or medium according to claim 1, wherein the cells are transformed cells.

14. A process for preparing a useful substance comprising culturing cells in the medium according to claim 1.

15. The process according to claim 14, wherein the useful substance is an IgG antibody and/or the cells are transformed cells.
